(19) United States
(12) Patent Application Publication
(10) Pub. No.: US 2021/0186637 A1
(43) Pub. Date: Jun. 24, 2021

(54) BENDING STRUCTURE AND FLEXIBLE TUBE FOR MEDICAL MANIPULATOR

(71) Applicant: NHK SPRING CO., LTD., Yokohama-shi, Kanazaw-ku (JP)

(72) Inventors: Shimpei KUROKAWA, Yokohama-shi, Kanagawa (JP); Yuki HOTODA, Yokohama-shi, Kanagawa (JP)

(21) Appl. No.: 16/755,116

(22) PCT Filed: Oct. 2, 2018
(86) PCT No.: PCT/JP2018/036882

§ 371 Date: Apr. 9, 2020

(30) Foreign Application Priority Data
Oct. 12, 2017 (JP) 2017-198853

(51) Int. Cl. A61B 34/00; B25J 18/06; F16H 21/44; A61B 34/30; A61B 17/29

(52) U.S. Cl. CPC A61B 34/71; B25J 18/06; A61B 2017/2902; A61B 34/30; A61B 17/29; F16H 21/44

(57) ABSTRACT

Provided are a bending structure and a flexible tube for a medical manipulator, which comprise a corrugated tube portion having a corrugated portion in which crests and troughs are alternately arranged in the axial direction and being bendable according to expansion and contraction of the crests and the troughs, and insertion holes provided on the corrugated portion to pass the drive wires in the axial direction.

### FIELD OF THE INVENTION

The present invention relates to a flexible tube and a bending structure applicable to a bendable part of a medical manipulator such as a surgical robot.

### BACKGROUND OF THE INVENTION

In recent medical treatment, a medical manipulator such as robot forceps for a surgical robot and manual forceps becomes broadly widened in order to enable to lighten burdens on both a patient and a doctor at the time of a surgery.

The medical manipulator such as the robot forceps and manual forceps allows a doctor to insert an arm as well as an endoscope camera through a small wound of a patient and perform a surgery with feeling as if forceps are actually manipulated while capturing a surgical field with eyes through a 3D monitor.

As such a medical manipulator, there is one which provides an arm with a joint function by means of a bendable part to secure a high degree of freedom and allow more fine surgical operation like Patent document 1.

In the medical manipulator, a coiled spring is used for the bendable part of the arm so that the coiled spring is bent by drawing drive wires passing through an inside thereof.

The arm of the medical manipulator is desired to be reduced in size in order to make a wound of a patient smaller and lighten mental and physical burdens. Accordingly, the bendable part used in the arm is also desired to be reduced in size.

In the technique of Patent document 1, however, the bendable part is composed of the coiled spring and therefore is limited on the seize reduction for necessity of securing load bearing and bendability.

Such a problem is existed in not only the above-mentioned medical manipulator such as the robot forceps and the manual forceps but also other types of medical manipulators such as an endoscope camera.
PATENT DOCUMENT 1: JP 2014-38075 A

### SUMMARY OF THE INVENTION

A problem to be solved is that there is a limit on securing load bearing and bendability while conducting size reduction.

In order to conduct size reduction and provide superior load bearing and bendability, the present invention is most characterized by a flexible tube through which drive wires for a medical manipulator are passed in an axial direction and being configured to be bent according to operation of the drive wires, comprising, a corrugated tube portion having a corrugated portion in which crests and troughs are alternately arranged in the axial direction and being bendable according to expansion and contraction of the crests and the troughs; and through portions provided on the corrugated portion to pass the drive wires in the axial direction.

Since the corrugated tube portion is bent according to the expansion and contraction of the crests and the troughs, the present invention makes it possible to obtain the flexible tube having superior load bearing and bendability while conducting size reduction.

Further, the present invention uses the corrugated tube portion as a guide for the drive wires by passing the drive wires through the through portions provided on the corrugated portion that comprises the crests and the troughs of the corrugated tube portion, and therefore the drive wires are retained at appropriate positions to stably and accurately conduct bending motion.

### BRIEF DESCRIPTION OF THE DRAWINGS

FIG. 1 is a perspective view illustrating robot forceps having a flexible tube according to an embodiment 1 of the present invention;

FIG. 2 is a front view of the robot forceps of FIG. 1;

FIG. 3 is a sectional view of the robot forceps of FIG. 1;

FIG. 4 is a perspective view of the partly omitted robot forceps of FIG. 1;

FIG. 5 is a side view of the partly omitted robot forceps of FIG. 1;

FIG. 6 is a sectional view of the partly omitted robot forceps of FIG. 1;

FIG. 7 is a perspective view of the flexible tube of the robot forceps of FIG. 1;

FIG. 8 is a front view of the flexible tube of FIG. 7;

FIG. 9(A) is a sectional view of the flexible tube of FIG. 7 and FIG. 9(B) is an enlarged view of a IX part of FIG. 9(A);

FIG. 10 is a sectional view of the flexible tube at the time of bending;

FIG. 11(A) is a graph illustrating a relationship between loads and bending angles of the flexible tube and FIG. 11(B) is a schematic view illustrating bending directions;

FIG. 12 is a perspective view illustrating a flexible tube according to an embodiment 2 of the present invention;

FIG. 13 is a side view of the flexible tube of FIG. 12;

FIG. 14 is a sectional view of the flexible tube of FIG. 12;

FIG. 15 is a sectional view illustrating robot forceps that uses a bending structure according to an embodiment 3 of the present invention;

FIG. 16 is a perspective view of the partly omitted robot forceps of FIG. 15;

FIGS. 17(A) and 17(B) are sectional views illustrating the bending structure of FIG. 15 in which FIG. 17(A) illustrates a normal state and FIG. 17(B) illustrates a bending state;

FIG. 18 is a graph illustrating relationships between loads and bending angles of bending structures;

FIG. 19 is a perspective view of partly omitted robot forceps using a bending structure according to an embodiment 4 of the present invention;

FIG. 20 is a sectional view of the robot forceps of FIG. 19;

FIG. 21 is a plan view of an elastic member according to a modification of the embodiment 4;

FIG. 22 is a plan view of an elastic member according to another modification of the embodiment 4;

FIG. 23 is a perspective view of partly omitted robot forceps using a bending structure according to an embodiment 5 of the present invention;

FIG. 24 is a sectional view of the robot forceps of FIG. 23; and

FIG. 25 is a perspective view of an elastic member used for the bending structure of FIG. 23.

### EMBODIMENT FOR CARRYING OUT THE INVENTION

The present invention accomplishes the object of conducting size reduction and providing superior load bearing and bendability by a flexible tube having through portions for passing drive wires that are formed with respect to a corrugated portion of a corrugated tube portion in which crests and troughs are alternately arranged in an axial direction.

The through portions are preferably provided in a circumferential direction of the corrugated tube portion and preferably have a constant distance from an axial center of the corrugated tube portion in a radial direction.

The through portions may be insertion holes each provided on a web between a crest and a trough of the corrugated tube portion being adjacent to each other in an axial direction. The through portions may be, however, insertion holes, cutouts, recessed portions or the like provided on the crests or the troughs.

The insertion hole may be positioned in the middle of an outer diameter at the crest and an inner diameter at the trough.

Further, an elastic member may be provided in the flexible tube to form a bending structure. The elastic member is configured to be arranged in the corrugated tube portion, have higher rigidity in the axial direction than the corrugated tube portion, and be bendable as well as the corrugated tube portion.

The elastic member may employ a variety of shapes and be, for example, a coiled spring, a solid cylinder, a hollow cylinder or the like located on an axial center portion of the corrugated tube portion.

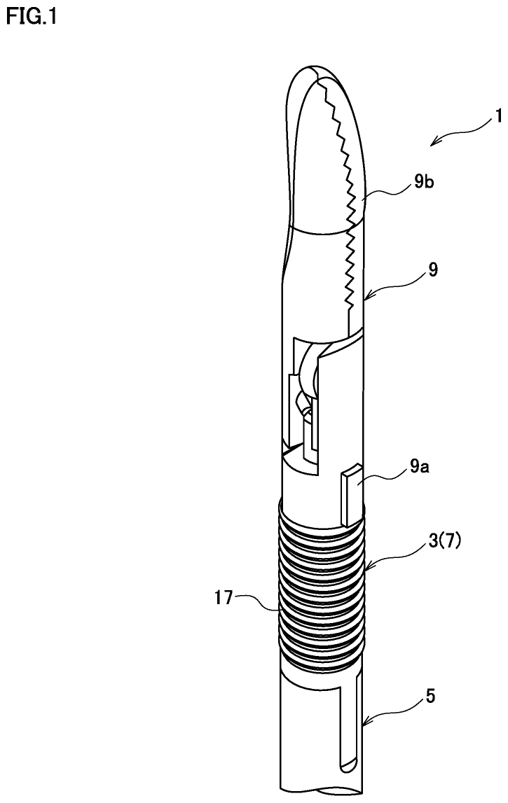

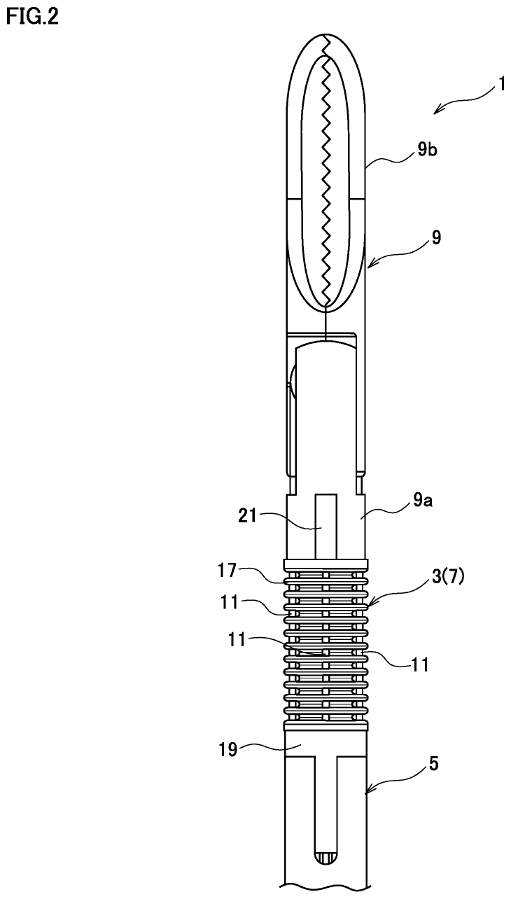

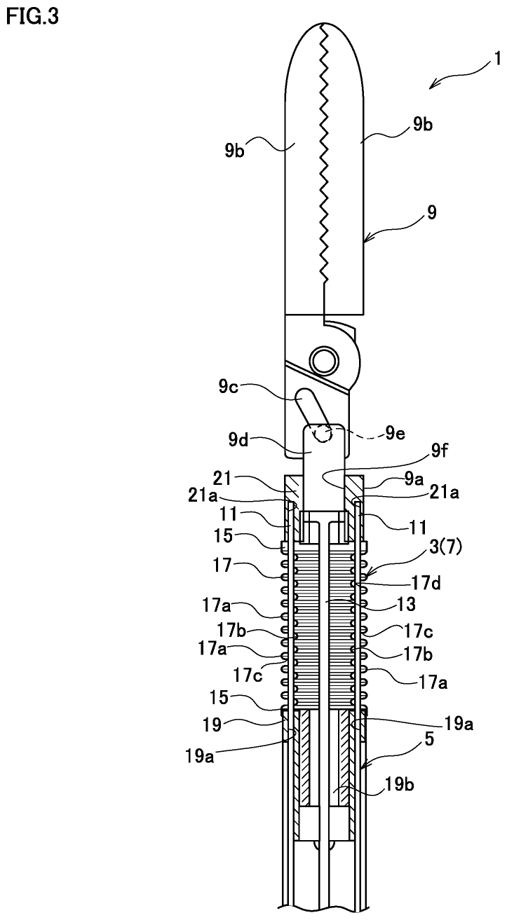

FIG. 1 is a perspective view illustrating robot forceps having a flexible tube according to the embodiment 1 of the present invention, FIG. 2 is a front view of the same, and FIG. 3 is a sectional view of the same.

Robot forceps 1 composes a front end of a robot arm of a surgical robot as a medical manipulator. In addition, the robot forceps 1 are an example of a medical manipulator.

It should be noted that a medical manipulator to which the flexible tube 3 is applicable is not limited particularly as long as, regardless of whether being attached to a surgical robot, one is manipulated by a hand of a doctor or the like and has a bendable part which performs bending motion.

The medical manipulator, therefore, also includes an endoscope camera, manual forceps and the like that are not attached to the surgical robot.

The robot forceps 1 of the present embodiment comprises a shaft part 5, a bendable part 7, and a grasping unit 9.

The shaft part 5 is formed into, for example, a cylindrical shape. Inside the shaft part 5, passings are drive wires 11 for driving the bendable part 7 and a push/pull cable 13 for driving the grasping unit 9. At a front end of the shaft part 5, the grasping unit 9 is provided through the bendable part 7.

The driving wires 11 are sufficient to be cord members, may be, for example, stranded wires, NiTi (Nickel-titanium) solid wires, piano wires, articulated rods, chains, strings, stitches, ropes or the like, but are not limited thereto.

The bendable part 7 comprises a flexible tube 3 according to the present embodiment. The bendable part 7 (flexible tube 3) passes the drive wires 11 and the push/pull cable 13 therethrough in an axial direction and is bendable according to operation of the driving wires 11. The axial direction means a direction along an axial center of the flexible tube 3, is not necessarily a direction being strictly parallel to the axial center, but also includes a direction slightly inclining relatively to the axial center.

In addition, the push/pull cable 13 is provided on an axial center portion of the bendable part 7 (flexible tube 3). The four driving wires 11 are provided so as to be located at 90 degrees in a circumferential direction according to the present embodiment, and are outwardly displaced and located relatively to the push/pull cable 13 in a radial direction, respectively. The details of the flexible tube 3 will be explained later. In addition, the radial direction is a radial direction of the flexible tube 3.

The grasping unit 9 has a pair of grasping parts 9b which are openably pivotally supported with a base part 9a attached to a front end of the bendable part 7. To the base part 9a, the drive wires 11 passing through the bendable part 7 are connected.

The grasping unit 9, therefore, is capable of orienting the grasping parts 9b to a desired direction while bending the bendable part 7 by operation of the driving wires 11.

To the grasping parts 9b, groove portions 9c are provided so as to be inclined relatively to the axial direction in a closed state of the grasping parts. Projections 9e of a movable piece 9d slidably engage with the groove portions 9c of the grasping parts 9b. The movable piece 9d is arranged in a through-hole 9f of the base part 9a of the grasping unit 9 movably in the axial direction and is connected to the push/pull cable 13 passing through the bendable part 7.

The grasping parts 9b, therefore, are configured to be opened and closed by the movable piece 9d moving in the axial direction according to reciprocating movement (push/pull movement) of the push/pull cable 13. It should be noted that the driving of the grasping unit 9 to open and close the grasping parts 9b is not limited to use of the push/pull cable 13 and may be used an air tube or drive cables.

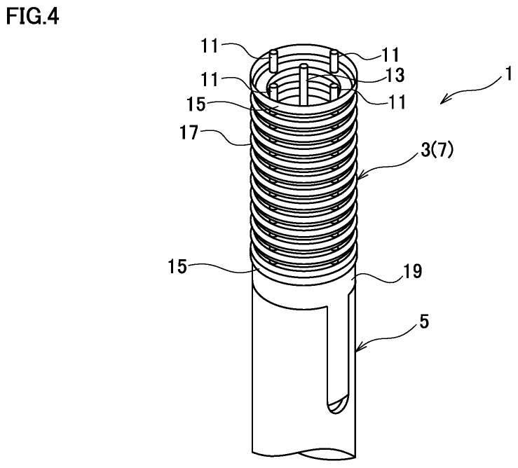

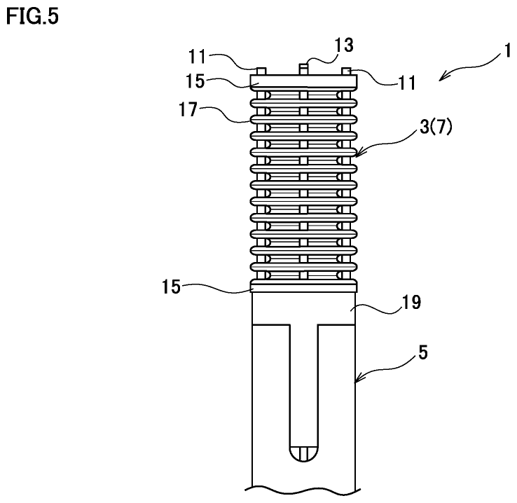

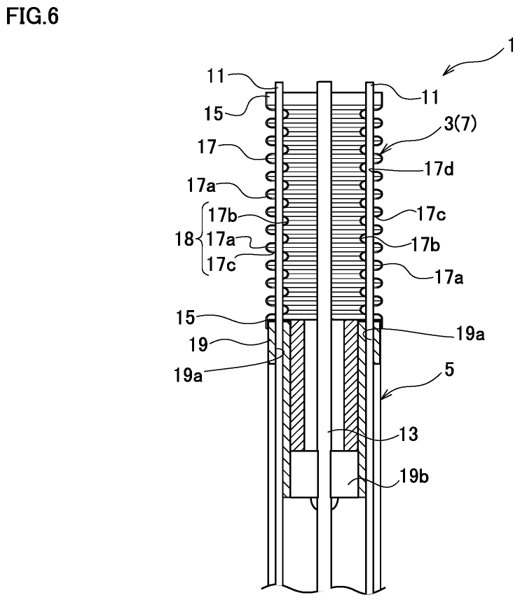

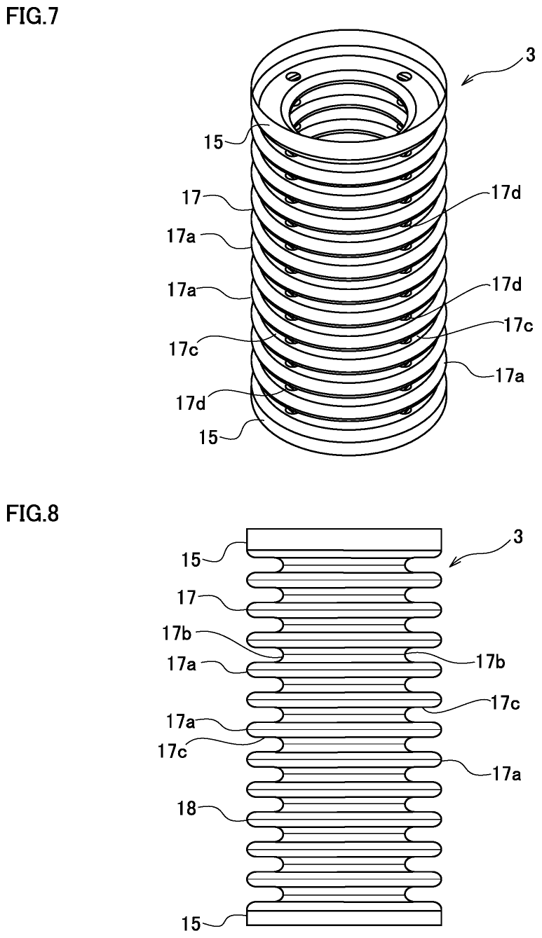

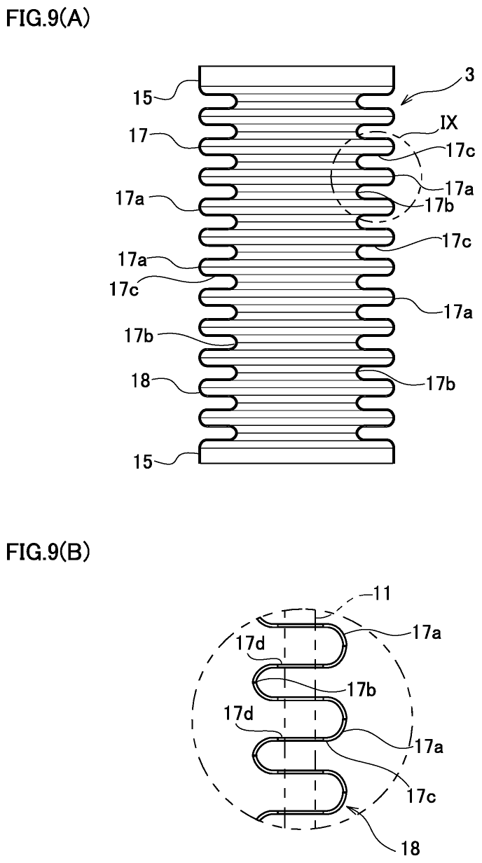

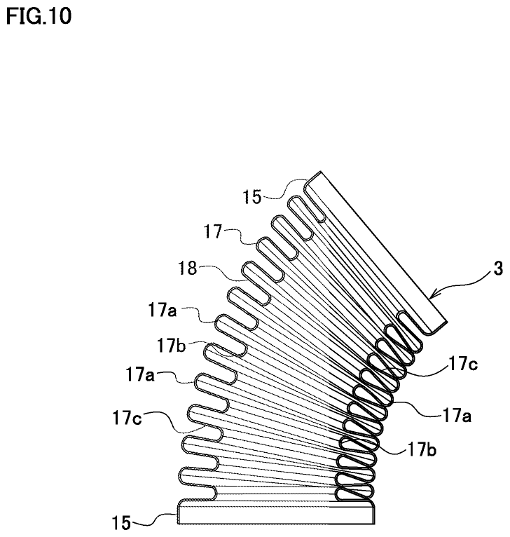

FIG. 4 is a perspective view of the partly omitted robot forceps 1 of FIG. 1, FIG. 5 is a side view of the same, and FIG. 6 is a sectional view of the same. FIG. 7 is a perspective view of the flexible tube 3, and FIG. 8 is a sideview of the same. Further, FIG. 9(A) is a sectional view of the flexible tube of FIG. 1 and FIG. 9(B) is an enlarged view of a IX part of FIG. 9(A). FIG. 10 is a sectional view of the flexible tube at the time of bending.

As illustrated in FIGS. 1-10, the flexible tube 3 is a bellows made of metal such as nickel and is formed into a tubular shape. It should be noted that the material of the flexible tube 3 may be appropriately employed according to required characteristics, manufacturing method or the like.

The flexible tube 3, as the bendable part 7 of the robot forceps 1, resiliently supports the grasping unit 9 relatively to the shaft part 5. According to the present embodiment, the flexible tube 3 comprises end tube portions 15, and a corrugated tube portion 17.

The end tube portions 15 are circular ring portions located at respective end of the flexible tube 3. The end tube portions 15 are respectively fitted to the front end side of the shaft part 5 and the base part 9a side of the grasping unit 9 of the robot forceps 1 to allow the flexible tube 3 to be attached to the robot forceps 1 side.

According to the present embodiment, the end tube portions 15 are fitted to a first connection part 19 and a second connection part 21 fixed to the front end of the shaft part 5 and the base part 9a of the grasping unit 9.

The first and second connection parts 19, 21 respectively composes parts of the front end of the shaft part 5 and the base part 9a of the grasping unit 9 and are formed of resin, metal or the like into cylindrical shapes.

In the first connection part 19, the driving wires 11 pass through through-holes 19a in the axial direction. In the second connection part 21, front end portions of the driving wires 11 are fixed into fixing holes 21a. Further, on an axial center portion of the first connection part 19, a cable passing hole 19b is provided to pass the push/pull cable 13 therethrough.

Between the end tube portions 15 of the flexible tube 3, the corrugated tube portion 17 is integrally provided.

The corrugated tube portion 17 is formed into a hollow circular tubular shape continuously transitioning from the end tube portions 15. In addition, the corrugated tube portion 17 and the end tube portions 15 may be configured to have the same thickness or different thicknesses. Further, thickness may vary among crests 17a, troughs 17b, and webs 17c of the corrugated tube portion 17 to be explained later.

The corrugated tube portion 17 has a corrugated portion 18 formed into a corrugated shape in which the crests 17a and the troughs 17b are alternately arranged according to variation in the diameter in the axial direction and being bendable according to expansion and contraction of the crests 17a and the troughs 17b.

In addition, the corrugated tube portion 17 may have a tubular shape such as square tube. In the case of the square tube, however, it is preferably that the corrugated tube portion 17 has a plan view such as foursquare, regular hexagon, or regular octagon being point symmetrical around an axial center of the corrugated tube portion in order to suppress anisotropy as explained later.

The crests 17a and the troughs 17b of the corrugated portion 18 have sectional shapes curved into arc shapes. Outer diameters of the crests 17a are constant and the same as outer diameters of the end tube portions 15. Pitches between the crests 17a and inner diameters of the troughs 17b are constant. The outer diameters of the crests 17a, the pitches between the crests 17a, and the inner diameters of the troughs 17b may be, however, varied in the axial direction.

Radii of curvatures of the crests 17 and the troughs 17b are the same as each other according to the present embodiment. The radii of curvatures may be, however, different from each other.

An interposition between the crest 17a and the trough 17b being adjacent to each other is the web 17c being flat in the radial direction. On the web 17c, an insertion hole 17d is formed as a through portion. Accordingly, the insertion holes 17d are formed on the corrugated portion 18 in the present invention. It should be noted that the insertion holes 17d may be formed on the crests 17a or the troughs 17b having the curved shape.

The corrugated shape of the corrugated portion 18 of the corrugated tube portion 17 is not limited particularly and may be formed into a sine wave, a triangular wave, a rectangular wave, or a sawtooth wave as a whole by, for example, setting of the sectional shapes of the crests 17a, the troughs 17b, and the webs 17c.

The insertion holes 17d are provided on each web 17c in the circumferential direction of the corrugated tube portion. According to the present embodiment, since the four drive wires 11 are provided respectively at 90 degrees in the circumferential direction, the four insertion holes 17d are provided at 90 degrees in the circumferential direction on each web 17c accordingly.

Between the webs 17c adjacent to each other in the axial direction, the insertion holes 17d are communicated with each other in the axial direction and the drive wires 11 are passed through the communicating insertion holes 17d. With the passing, the flexible tube 3 passes the drive wires 11 therethrough in the axial direction as the through portions and functions as a guide to retain the drive wires at given positions.

It should be noted that the through portions may be cutouts or recessed portions radially recessed from an inner periphery or outer periphery of the main body 15 of the flexible tube 3 instead of the insertion holes 17d. The flexible tube 3 may, therefore, axially pass the drive wires 11 alongside the through portions being the recessed portions or the like on the inner periphery or outer periphery.

Further, each insertion hole 17d is positioned in the middle of the outer diameter at the crest 17a and the inner diameter at the trough 17b on the web 17c. The insertion hole 17d may be, however, displaced radially inwardly or outwardly with respect to the middle of the outer diameter and the inner diameter. Further, distances from the axial center of the main body 15 to the respective insertion holes 17d may be appropriately set according to the characteristics of the flexible tube 3 and, for example, may or may not be constant.

A shape of the insertion hole 17d is circular, the diameter of which is larger than the diameter of the drive wire 11. The difference between the diameters permits the expansion and contraction of the crests 17a and the trough 17b at the time of the bending of the flexible tube 3. It should be noted that the shape of the insertion hole 17d is not limited to the circular shape and may be another shape such as rectangular shape as long as the expansion and contraction of the crests 17a and the trough 17b are permitted.

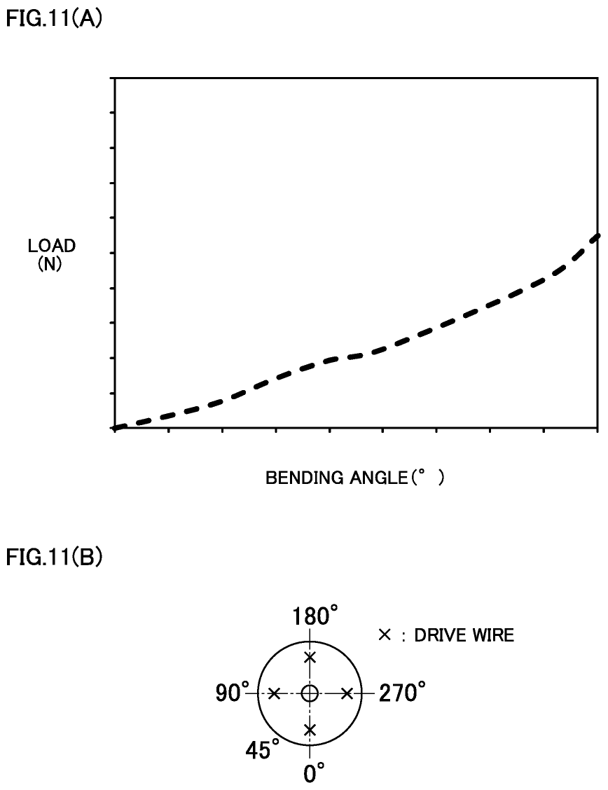

In the flexible tube 3 as the bendable part 7, the movable side located on the grasping unit 9 side bends relatively to the stationary side located on the shaft part 5 side as illustrated in FIG. 10 by drawing any one of the drive wires 11 (FIG. 11(B)) when a doctor manipulates the robot forceps 1. Then, a number of the drive wires 11 are combined to be drawn, thereby to allow the flexible tube to bend omnidirectionally at 360 degrees.

When drawing any one of the drive wires 11 to conduct the bending, the flexible tube 3 is compressed at the crests 17a and the troughs 17b on an inner portion of the bending relative to a neutral axis and is extended at the crests 17a and the troughs 17b on an outer portion of the bending relative to the neutral axis.

Namely, the crests 17a and the troughs 17b on the inner portion of the bending deforms so as to shrink a spread in the axial direction, and the crests 17a and the troughs 17b on the outer portion of the bending deforms so as to enlarge a spread in the axial direction.

By deforming in this way, the flexible tube 3 bends as a whole. Such bending motion is performed omnidirectionally at 360 degrees in the same way as the above without a change in the bending state, thereby to suppress anisotropy.

Further, at the time of the bending, the flexible tube 3 passes the drive wires 11 through the insertion holes 17d to retain the drive wires at the appropriate positions, so that the flexible tube 3 stably and accurately conducts the bending motion according to the manipulation of the doctor.

Furthermore, although the drive wires 11 bend according to the bending of the flexible tube 3, the drive wires pass through each web 17c that displaces so as to incline according to the bending of the flexible tube 3 at this time, thereby to secure stability of the manipulation.

FIG. 11(A) is a graph illustrating a relationship between loads and bending angles of the flexible tube 3 according to the embodiment 1 and FIG. 11(B) is a schematic view illustrating bending directions.

In FIG. 11(A), loads are plotted when any one of the drive wires 11 of FIG. 11(B) is operated to bend the flexible tube 3 toward said any one of the drive wires 11 (0 degree, 90 degrees, 180 degrees, or 270 degrees in FIG. 11(B)) from 0 degree to 90 degrees in a bending angle.

As illustrated in FIG. 11(A), the linearity of the increase in load relative to the increase from 0 degree to 90 degrees in a bending angle is high and therefore the load bearing and the bendability are superior.

As mentioned above, the flexible tube 3 according to the present embodiment is provided with the corrugated tube portion 17 having the corrugated portion 18 in which the crests 17a and the troughs 17b are alternately arranged in the axial direction and being bendable according to the expansion and contraction of the crests 17a and the troughs 17b, and the insertion holes 17d as the through portions provided on the corrugated portion 18 to pass the drive wires 11 in the axial direction.

Since the corrugated tube portion 17 is bent according to the expansion and contraction of the crests 17a and the troughs 17b, therefore, the present embodiment heightens the linearity of the load bearing of the bending angles and the loads. The present embodiment makes it possible to obtain the flexible tube 3 having the superior load bearing and bendability while conducting size reduction.

Further, the present embodiment substantially uniforms the bending state of the crests 17a and the troughs 17b regardless of the bending direction and suppresses the anisotropy of the bending.

As a result, the flexible tube 3 stably and accurately conducts the bending motion according to the operation of the doctor.

Furthermore, the present embodiment passes the drive wires 11 through the corrugated portion 18 of the corrugated tube portion 17, thereby to use the corrugated tube portion 17 as the guide for the drive wires 11.

The present embodiment, therefore, retains the drive wires 11 at the appropriate positions to more stably and accurately conduct the bending motion.

Further, the flexible tube 3 has the high airtightness to prevent the inside thereof from being contaminated.

Further, the flexible tube 3 provides superior torsional rigidity.

According to the present embodiment, the insertion hole 17d is formed on the web 17c between the crest 17a and the trough 17b, so that the drive wires 11 are passed through the webs 17c inclined according to the bending of the flexible tube 3 to secure the stability of the operation of the drive wires 11.

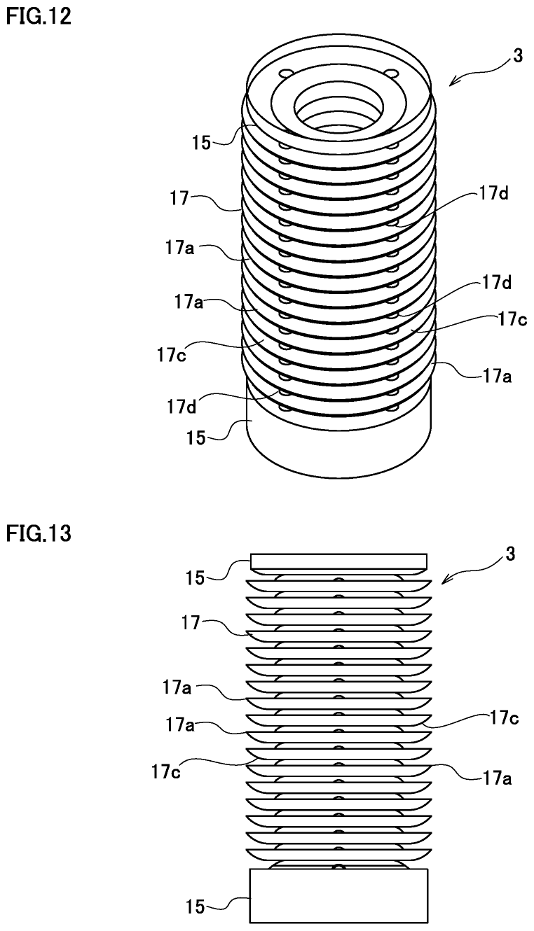

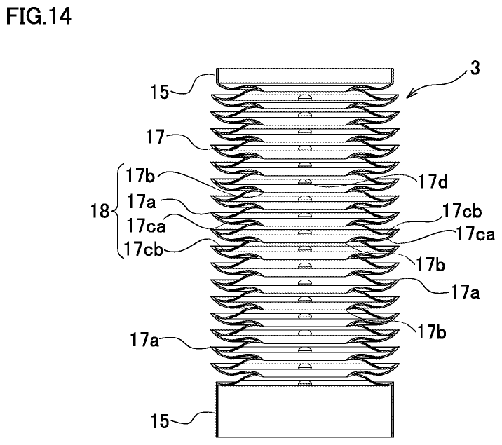

FIG. 12 is a perspective view illustrating a flexible tube according to the embodiment 2 of the present invention, FIG. 13 is a side view of the same, and FIG. 14 is a sectional view of the same. In addition, components in the embodiment 2 corresponding to those in the embodiment 1 are represented with the same numerals to eliminate duplicate explanation.

In the flexible tube 3 according to the present embodiment, a corrugated shape of a corrugated tube portion 17 is altered.

Namely, each crest 17a of the corrugated tube portion 17 has a sectional shape being a wedge shape in which a web 17ca on one side in an axial direction and a web 17cb on the other side are connected to each other. Each trough 17b has a sectional shape being a wedge shape in which, with reverse of the one side and the other side in the axial direction relative to the case of the crest 17a, the web 17cb on one side and the web 17ca on the other side are connected to each other.

The sectional shapes of the webs 17ca and 17cb are substantially the same shape curved in a cubic curve. In addition, the web 17cb is inclined relatively to the web 17ca.

With the inclination and the curved shape, part of the web 17cb is located within a length of the web 17ca in the axial direction. Namely, the part of the web 17cb and the part of the web 17ca overlap each other in a radial direction.

The corrugated tube portion 17 of the present embodiment is, therefore, reduced in length as a whole.

Further, in each web 17ca, part on a radial inner side and part on a radial outer side overlap each other in the radial direction. By that amount, the corrugated tube portion 17 is reduced in length in the axial length.

Accordingly, the present embodiment reduces the corrugated tube portion 17 in size in the axial direction. In addition, the present embodiment also provides the same effect as the embodiment 1.

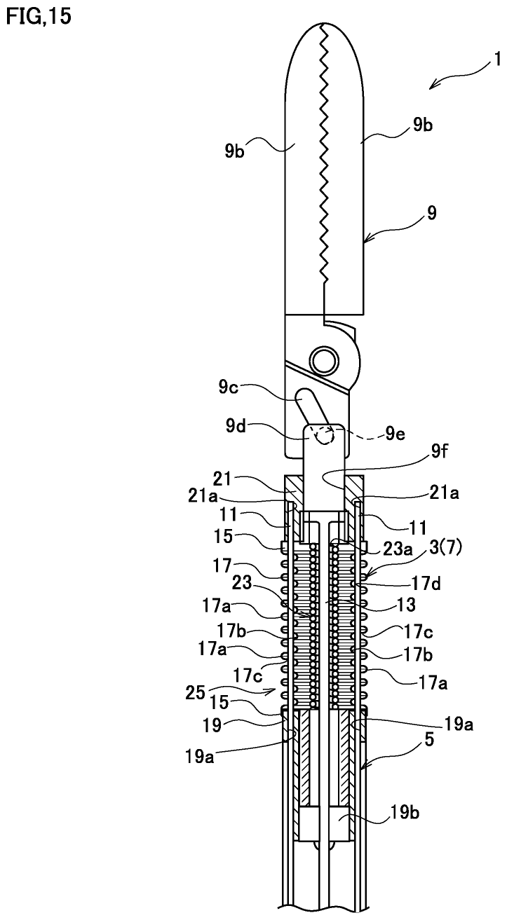

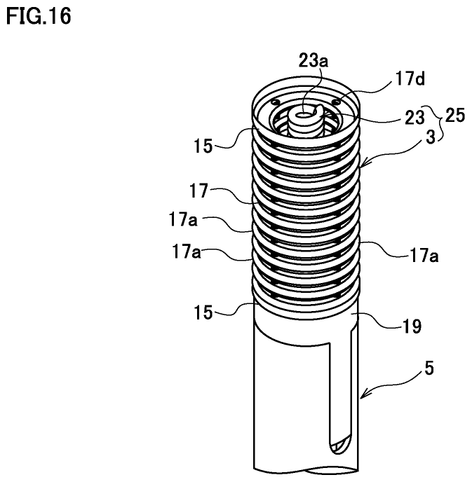

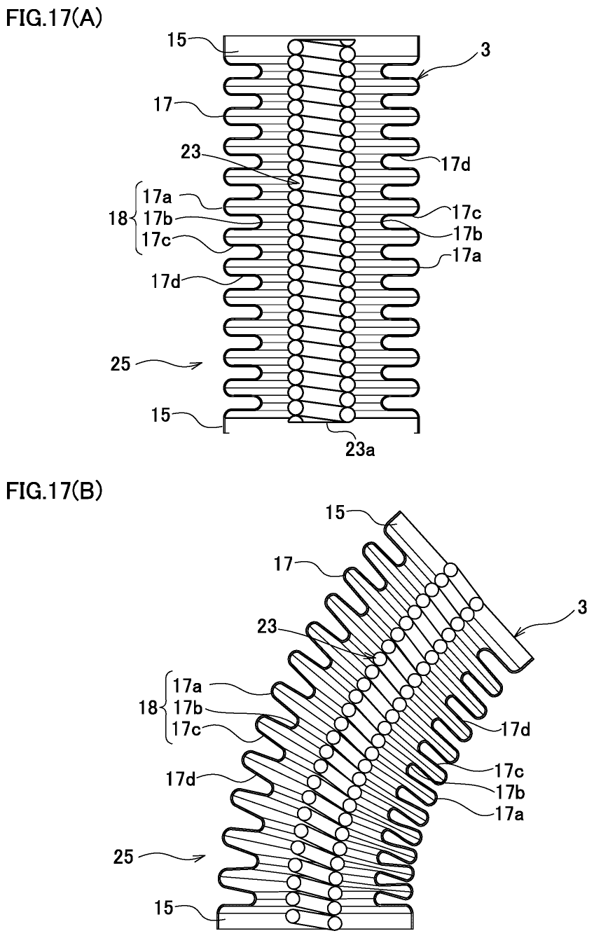

FIG. 15 is a sectional view illustrating robot forceps that uses a bending structure having a flexible tube according to the embodiment 3 of the present invention, and FIG. 16 is a perspective view of the partly omitted robot forceps of FIG. 15. Further, FIG. 17 is a set of sectional views illustrating the bending structure of FIG. 15 in which FIG. 17(A) illustrates a normal state and FIG. 17(B) illustrates a bending state. In addition, components in the embodiment 3 corresponding to those in the embodiment 1 are represented with the same numerals to eliminate duplicate explanation.

According to the present embodiment, an elastic member 23 is arranged in the flexible tube 3 of the embodiment 1 to form a bending structure 25.

The elastic member 23 is a coiled spring made of metal, in particular a close contact coiled spring. In addition, the close contact coiled spring means a coiled spring in which coils are in closely contact with each other in a free state. As the elastic member 23, a non-close contact coiled spring may be used, the non-close contact coiled spring having a gap between coils in a free state.

The elastic member 23 of the present embodiment has a sectional shape of an element wire of the coiled spring being circular. The sectional shape of the element wire of the coiled spring may be, however, another shape such as rectangular or oval shape.

The elastic member 23 is arranged on the axial center portion of the flexible tube 3 so as to define a cable insertion hole 23a through which a push/pull cable 13 passes on an inner periphery. An outer periphery of the elastic member 23 has a gap with respect to troughs 17b of the flexible tube 3.

In an axial direction, the elastic member 23 extends over at least a whole corrugated tube portion 17 of the flexible tube 3, rigidity against compression of which is set higher than that of the flexible tube 3. Accordingly, the elastic member 23 is capable of preventing the flexible tube 3 from being unexpectedly compressed in the axial direction.

Further, the elastic member 23 is bendable according to the corrugated tube portion 17 and has a function to adjust load characteristics of the flexible tube 3 according to load characteristics in a bending direction.

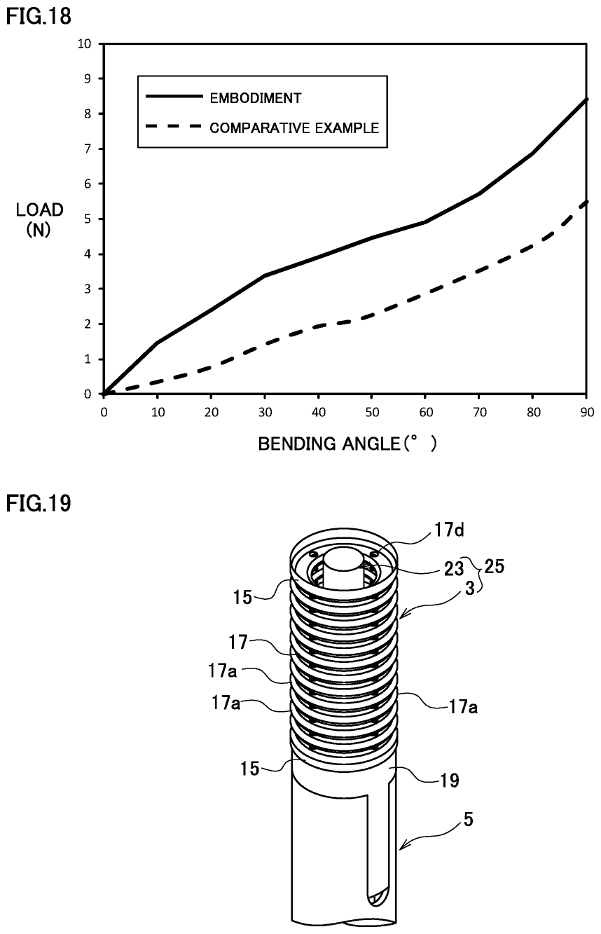

FIG. 18 is a graph illustrating relationships between loads and bending angles of the bending structures 25 according to the embodiment 3 and a comparative example.

As the comparative example, the relationship between the loads and the bending angles of the flexible tube 3 of the embodiment 1 is illustrated.

The embodiment 3, similar to the comparative example, loads are plotted when the bending structure 25 is bent from 0 degree to 90 degrees in the bending angle.

The embodiment 3 heightens the loads all over the bending angles relatively to the comparative example while maintaining linearity of increase of the loads relative to increase from 0 degrees to 90 degrees in the bending angle, so that load bearing and bendability are superior.

As mentioned above, the bending structure 25 of the present embodiment is provided with the elastic member 23 being arranged in the corrugated tube portion 17 of the flexible tube 3, having the higher rigidity in the axial direction than the corrugated tube portion 17, and being bendable according to the bending of the corrugated tube portion 17.

The bending structure 25 of the present embodiment is, therefore, capable of preventing the flexible tube 23 from being unexpectedly compressed.

Accordingly, although behavior of the bendable part 7 in response to operation of driving wires 11 may be unstable if the flexible tube 3 is unexpectedly compressed, the present embodiment suppresses such unstable behavior. Further, a path length is not varied at the time of the bending, so that operation of the grasping unit 9 is stable.

Further, the bending structure 25 of the present embodiment adjusts the load characteristics of the flexible tube 3 according to the load characteristics in the bending direction of the elastic member 23.

In addition, the present embodiment provides the same effects as the embodiment 1.

It should be noted that the elastic member 23 is applicable to the embodiment 2.

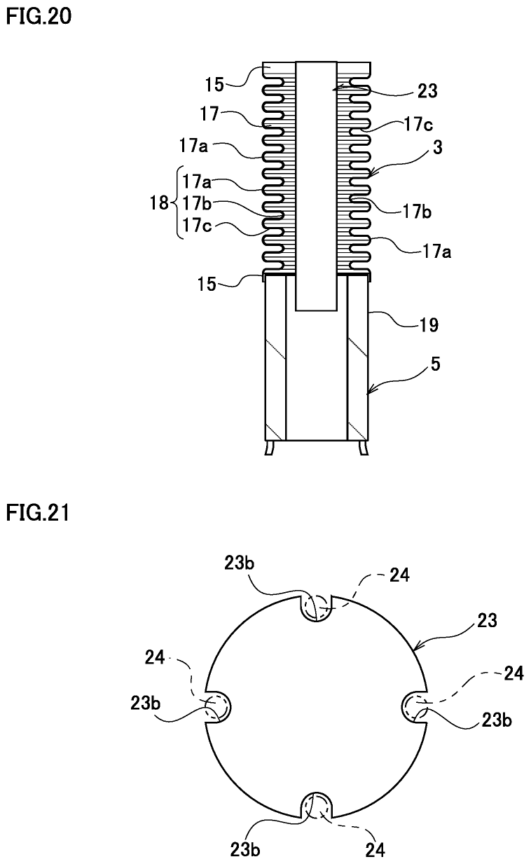

FIG. 19 is a perspective view of partly omitted robot forceps that is provided with a bending structure according to the embodiment 4 of the present invention, and FIG. 20 is a sectional view of the same. In addition, components in the embodiment 4 corresponding to those in the embodiment 3 are represented with the same numerals to eliminate duplicate explanation.

A bending structure 25 of the present embodiment is what an elastic member 23 is made solid cylindrical. The others are the same components as the embodiment 3.

The elastic member 23 is formed of elastic material such as rubber into a solid cylinder. With this, the elastic member 23 is configured to have higher rigidity in an axial direction than a main body 15 of a flexible tube 3 and be bendable according to bending of the flexible tube 3.

In addition, in the present embodiment, since the solid cylindrical elastic member 23 is located on an axial center portion of the flexible tube 3, drive wires or the like are preferably employed instead of a push/pull cable 13 to drive a grasping unit 9.

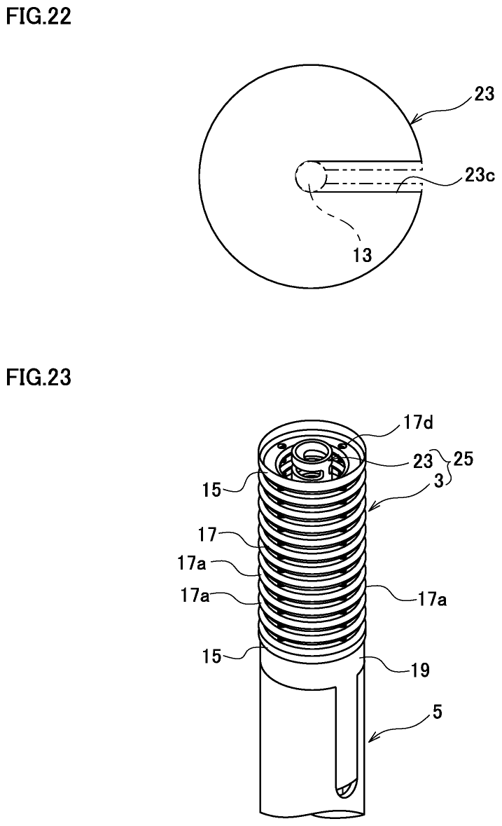

FIG. 21 a plan view illustrating an elastic member 23 according to a modification, and FIG. 22 is a plan view illustrating an elastic member 23 according to another modification.

The modification of FIG. 21 forms groove portions 23b on an outer periphery of the solid cylindrical elastic member 23, the groove portions being recessed in a radial direction. The groove portions 23b are provided along the elastic member 23 in the axial direction, to guide drive wires 24 employed instead of the push/pull cable 13 for driving the grasping unit 9.

It should be noted that the number and the arrangement of the drive wires 24 are appropriately altered according to the structure of the grasping unit 9, and accordingly the number and the arrangement of the groove portions 23b are appropriately altered.

The modification of FIG. 22 provides the solid cylindrical elastic member 23 with a slit 23c being recessed from an outer periphery to the vicinity of an axial center portion in the radial direction. The slit 23c is provided along the elastic member 23 in the axial direction to guide the push/pull cable 13 in the axial center portion of the elastic member 23.

In addition, the slit 23c, as illustrated with a two-dot chain line, may be configured to be slightly narrower than a diameter of the push/pull cable 13 from the outer periphery of the elastic member 23 before the axial center portion and have the same diameter as the push/pull cable 13 at the axial center portion. Further, the slit 23c may be provided so as to pass across the axial center portion of the elastic member 23.

The embodiment 4 and the modifications provide the same effects as the embodiment 3.

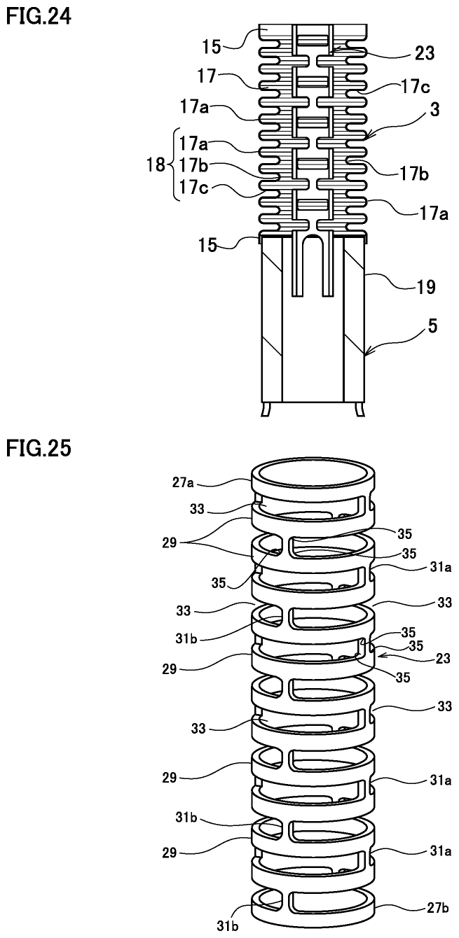

FIG. 23 is a perspective view of partly omitted robot forceps being provided with a bending structure according to the embodiment 5 of the present invention, FIG. 24 is a sectional view of the same, and FIG. 25 is a perspective view illustrating an elastic member used for the bending structure of FIG. 24. In addition, components in the embodiment 5 corresponding to those in the embodiment 3 are represented with the same numerals to eliminate duplicate explanation.

A bending structure 25 of the present embodiment is what an elastic member 23 is made into a hollow cylinder. The others are the same components as the embodiment 3.

The elastic member 23 is made of super elastic alloy and comprises end tube portions 27a, 27b, ring portions 29, tube connecting portions 31a, 31b, and tube slits 33. In addition, the super elastic alloy may be NiTi alloy (Nickel-titanium alloy), titanium-base alloy such as gummetal (registered trademark), Cu—Al—Mn alloy (copper-base alloy), Fe—Mn—Al alloy (iron-base alloy) or the like.

The end tube portions 27a, 27b are rings provided at respective end portions. Between the end tube portions 27a, 27b, the ring portions 29 are located.

The ring portions 29 are successively parallelly provided at regular intervals in an axial direction. Spreads in the axial direction of the ring portions 29 are constant according to the embodiment. The spreads in the axial direction of the ring portions 29 may be, however, gradually reduced from a stationary side located on a shaft portion 5 side to a movable side located on a grasping unit 9 side.

The adjacent ring portions 29 are connected by the tube connecting portions 31a, 31b at parts in a circumferential direction. The ring portions 29 at respective ends are connected by the tube connecting portions 31a, 31b to the end tube portions 27a, 27b.

The tube connecting portions 31a, 31b are provided integrally to the ring portions 29 and connects the ring portions 29 being adjacent to each other in the axial direction at two parts in the circumferential direction, the two parts opposing each other in a radial direction.

In each ring portion 29, the tube connecting portions 31a, 31b located on one side (base end side) in the axial direction and the tube connecting portions 31a, 31b located on the other side (front end side) in the axial direction are arranged to be displaced by 180/N degrees in the circumferential direction.

The displacement of the tube connecting portions 31a, 31b means displacement between center lines of the tube connecting portions 31a, 31b (the same shall apply hereinafter). N is an integer equal to or more than 2. According to the present embodiment, N=2 is set and the tube connecting portions 31a, 31b are arranged to be displaced by 90 degrees.

It should be noted that the displacement between the tube connecting portions 31a, 31b may be 60 degrees or the like, but is preferably 90 degrees. This reduces the number of the ring portions 29 required to bend the flexible tube 3 and makes the entire length compact.

Each tube connecting portion 31a, 31b is a rectangular plate extended in the axial direction and has a slight curvature according to the ring portion 29. Widths of the tube connecting portions 31a, 31b in the circumferential direction are constant according to the present embodiment and may be gradually reduced from the stationary side located on the shaft portion 5 side to the movable side located on the grasping unit 9 side.

If the widths of the tube connecting portions 31a, 31b in the circumferential direction are gradually reduced toward the movable side, the spread of the ring portions 29 in the axial direction may be smaller than the maximum width of the connecting tube portion 31a, 31b in the circumferential direction. In this case, the minimum width of the tube connecting portion 31a, 31b in the circumferential direction is preferably equal to the spread of the ring portions 29 in the axial direction.

Both ends of the tube connecting portions 31a, 31b in the axial direction transition through arc portions 35 to the ring portions 29. Accordingly, the tube connecting portions 31a, 31b and the ring portions 29 are tangentially continued to each other.

In addition, the tube connecting portions 31a, 31b and the ring portions 29 are transition to each other with no step on respective inner and outer peripheries in the radial direction of the ring portions 29. The tube connecting portions 31a, 31b may have, however, form to be thicker or thinner than the ring portions 29 to have steps.

The tube connecting portions 31a, 31b bend so that one side of a neutral axis as a boundary in the circumferential direction is compressed and the other side in the circumferential direction is extended, to allow the flexible tube 3 to be bent. According to the present embodiment, the tube connecting portions 31a, 31b displaced by 90 degrees in the circumferential direction are bent to allow bending in two different orthogonal directions.

On both sides of each tube connecting portion 31a, 31b in the circumferential direction, the tube slits 33 are provided to allow the bending of the flexible tube 3 based on the bending of the tube connecting portions 31a, 31b.

Namely, the tube slits 33 are defined on both sides of the tube connecting portions 31a, 31b in the circumferential direction between the ring portions 29 being adjacent to each other in the axial direction. Each tube slit 33 is a rectangular shape with rounded corners according to the shapes of the ring portions 29 and the tube connecting portions 31a, 31b.

The embodiment 5 also provides the same effects as the embodiment 3.

Further, according to the embodiment 5, the elastic member 23 made of the super elastic alloy is formed by connecting the ring portions 29 to each other with the tube connecting portions 31a, 31b in the axis direction and is bendable according to the bending of the tube connecting portions 31a, 31b, thereby to conduct size reduction and provide the superior load bearing and bendability.

Based on the characteristics, the present embodiment improves the characteristics of the whole bending structure 25.

Further, the elastic member 23, with the structure to connect the ring portions 29 by the tube connecting portions 31a, 31b, is superior in torsional rigidity. Accordingly, the present embodiment improves the torsional rigidity of the whole bending structure 25.

1. A flexible tube through which drive wires for a medical manipulator are passed in an axial direction and being configured to be bent according to operation of the drive wires, comprising:
a corrugated tube portion having a corrugated portion in which crests and troughs are alternately arranged in the axial direction and being bendable according to expansion and contraction of the crests and the troughs; and
through portions provided on the corrugated portion to pass the drive wires in the axial direction.

2. The flexible tube according to claim 1, wherein
the through portion is an insertion hole provided on a web between the crest and the trough of the corrugated tube portion being adjacent to each other in the axial direction.

3. The flexible tube according to claim 2, wherein
the insertion hole is positioned in a middle of an outer diameter at the crest and an inner diameter at the trough.

4. A bending structure being provided with the flexible tube according to claim 1, comprising:
an elastic member arranged in the corrugated tube portion, having higher rigidity in the axial direction than the corrugated tube portion, and being bendable as well as the corrugated tube portion.

5. The bending structure according to claim 4, wherein
the elastic member is a coiled spring, a solid cylinder, or a hollow cylinder located on an axial center portion of the corrugated tube portion.
